(19) United States
(12) Patent Application Publication
(10) Pub. No.: US 2017/0296444 A1
(43) Pub. Date: Oct. 19, 2017

(54) COSMETIC EMULSION SPRAY

(71) Applicant: BEIERSDORF AG, Hamburg (DE)

(72) Inventors: Kerstin SKUBSCH, Prisdorf (DE); Kaja LUETTIG, Hamburg (DE); Claudia MUELLER, Tangstedt (DE); Regine WERNER, Bienenbuettel (DE)

(73) Assignee: BEIERSDORF AG, Hamburg (DE)

(21) Appl. No.: 15/516,697

(22) PCT Filed: Sep. 25, 2015
(86) PCT No.: PCT/EP2015/072078

§ 371 Date: Apr. 4, 2017

(30) Foreign Application Priority Data
Oct. 9, 2014 (DE) 10 2014 220 452.6

(51) Int. Cl. A61K 8/04; A61K 8/92; A61K 8/891; A61K 8/81; A61K 8/37; A61K 8/34; B65D 83/62; A61K 8/06

(52) U.S. Cl. CPC A61K 8/046; B65D 83/62; A61K 8/922; A61K 8/92; A61K 8/891; A61K 8/062; A61K 8/37; A61K 8/347; A61K 8/345; A61K 8/34; A61K 8/8152

(57) ABSTRACT

The invention relates to a cosmetic spray which consists of a) an oil-in-water emulsion (O/W emulsion) that contains i) polyglyceryl-3 methylglucose distearate and ii) acrylate/C10-30 alkyl acrylate crosspolymer, and b) a spray applicator system.

The present invention relates to a cosmetic spray consisting of an oil-in-water emulsion (O/W emulsion) containing polyglyceryl-3 methylglucose distearate and acrylate/C10-30 alkyl acrylate crosspolymer and also a spray applicator system.

The desire to appear beautiful and attractive is naturally rooted in humans. Even if the beauty ideal has changed over the course of time, striving after a flawless appearance has always been the aim of humans. An essential part of a beautiful and attractive appearance is the condition and appearance of the skin.

Skincare products generally consist of emulsions. Emulsions are generally understood to mean heterogeneous systems which consist of two liquids immiscible, or of only limited miscibility, with each other, which are typically referred to as phases and in which one of the two liquids is dispersed in the form of fine droplets in the other liquid. Externally and with the naked eye, emulsions appear homogeneous.

If the two liquids are water and oil and oil droplets are present finely divided in water, it is an oil-in-water emulsion (O/W emulsion, e.g. milk). The basic character of an O/W emulsion is characterized by the water. In a water-in-oil emulsion (W/O emulsion, e.g. butter), the principle is reversed and the basic character is determined here by the oil.

The abundance of commercially available cosmetic emulsions should not however obscure the fact that these preparations of the prior art have a series of disadvantages.

Particularly when these emulsions are applied directly onto the skin using a spray applicator spray applicator system (e.g. an aerosol can or a bag-on-valve system, see below) from a reservoir under pressure, the problem arises that the preparations on the one hand should be stable to temperature and on storage and not prone to premature phase separation and on the other hand must be sufficiently thin to be sprayable at all.

Furthermore, in these preparations which are released by positive pressure, the problem always arises that the spray mist generated with the aid of a dispenser should have two apparently contradictory properties: on the one hand, the spray mist droplets should be as small as possible and finely dispersed and on the other hand the spray mist should be overall relatively well focused when exiting the system to enable targeted application to defined skin areas. These properties are, of course, influenced firstly by the configuration of the spray head and the dispenser. Secondly however, the composition and its rheological properties also have an influence on the droplet size and the “flight behavior” of the spray mist. Conventional preparations of the prior art have the disadvantage that the preparations emerge from the applicator system either in the form of a jet (“like a shot from a water pistol”), or that the spray mist is so finely dispersed or so heterogeneous in its droplet size that only a fraction of the preparation reaches the intended target, while a relatively large fraction of the droplets takes a different flight path and, inter alia, sinks prematurely to the ground. This then leads to an undesirable contamination of the application environment with the preparation during the application of the cosmetic.

It was therefore the object of the present invention to eliminate the deficiencies of the prior art and to develop a cosmetic spray whose spray pattern enables a focused fine spray mist without causing greater “scattering losses”. In addition, the sprayed preparation should on striking the skin adhere well thereto and not bounce off it again.

The object, surprisingly, is achieved by a cosmetic spray consisting of

a) an oil-in-water emulsion (O/W emulsion) containing
i) polyglyceryl-3 methylglucose distearate and
ii) acrylate/C10-30 alkyl acrylate crosspolymer and also
b) a spray applicator system.

It is preferred according to the invention in this case if the spray applicator system used is a bag-on-valve applicator system, in which a bag containing the O/W emulsion is in a pressurized gas container under positive pressure.

In the case of these bag-on-valve systems, the contents of the bag (in this case the O/W emulsion) is pressed outwards by the spray head by the positive pressure in the positive pressure container on opening the spray head and divided into small droplets (“spray mist”) by the dispenser present in the spray head. The pressure compensation therefore does not occur by the direct escape of the pressurized gas from the positive pressure container, but through emptying the contents of the storage bag.

It is advantageous according to the invention if the positive pressure in the pressurized gas container of the bag-on-valve applicator system is from 2 to 12 bar (based on the ambient pressure of 1.013 bar).

It is advantageous in accordance with the invention if the spray head of the spray applicator has a uniform spray jet over the entire lifetime of the can. From a distance of 10 cm, a spray pattern of 5-6 cm is favored. If the pressure falls over the entire lifetime of the can, the spray pattern should not exceed 8 cm.

According to the invention, advantageous embodiments of the present invention are characterized in that the bag containing the O/W emulsion is formed from a laminate of PE/adhesive/PA/adhesive/AL/adhesive/PET.

Particularly preferred according to the invention is a spray applicator with the following specification: 9 bar positive pressure with nitrogen valve DU 2527 or DU 3527 from Aptar®, BOV—cup: Alu gold lacquered—inner gasket: Buna KA 6712—body valve: PP—spring: Inox 302—piston: POM external gasket: butyl 1.2 mm foil: PET12/ALU8/OPA15/PP75.

In the context of the present invention, it is advantageous if the O/W emulsion according to the invention contains from 0.1 to 2% by weight polyglyceryl-3 methylglucose distearate, based on the total weight of the emulsion.

In the context of the present invention, it is preferred if the O/W emulsion according to the invention comprises from 0.5 to 1.5% by weight polyglyceryl-3 methylglucose distearate, based on the total weight of the emulsion.

In the context of the present invention, it is advantageous if the O/W emulsion according to the invention contains from 0.02 to 0.2% by weight acrylate/C10-30 alkyl acrylate crosspolymer, based on the total weight of the emulsion.

It is preferred in the context of the present invention, if the O/W emulsion according to the invention contains from 0.05 to 0.15% by weight acrylate/C10-30 alkyl acrylate crosspolymer, based on the total weight of the emulsion.

It is advantageous in accordance with the invention if the emulsion contains capryl/capric triglyceride, isopropyl palmitate and/or shea butter.

If the emulsion contains capryl/capric triglyceride, it is advantageous in accordance with the invention if this substance is present in this at a concentration of 1 to 7% by weight, based on the total weight of the emulsion.

If the emulsion contains isopropyl palmitate, it is advantageous in accordance with the invention if this substance is present in this at a concentration of 1 to 7% by weight, based on the total weight of the emulsion.

If the emulsion contains shea butter, it is advantageous in accordance with the invention if this substance is present in this at a concentration of 0.5 to 3% by weight, based on the total weight of the emulsion.

According to the invention, advantageous embodiments of the present invention are also characterized in that the emulsion contains dimethicone and/or cyclomethicone.

The oil phase of the emulsion according to the invention may also comprise further oil, fat and wax components, for example, polar oils from the group of the lecithins or compounds such as cocoglyceride, olive oil, sunflower oil, jojoba oil, soybean oil, peanut oil, rapeseed oil, almond oil, palm oil, coconut oil, castor oil, wheat germ oil, grape seed oil, safflower oil, evening primrose oil, macadamia nut oil and the like. It is also possible to use compounds such as phenethyl benzoate, 2-phenylethyl benzoate, isopropyl lauroyl sarcosinate, phenyl trimethicone, cyclomethicone, dibutyl adipate, octyl palmitate, octyl cocoate, octyl isostearate, octyldodecyl myristate, octyldodecanol, cetearyl isononanoate, isopropyl myristate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate.

Also advantageous in accordance with the invention are, for example, natural waxes of animal and vegetable origin such as beeswax and other insect waxes and berry wax, shea butter and/or lanolin (wool wax).

The oil phase may also be selected advantageously from the group of dialkyl ethers and dialkyl carbonates, e.g. dicaprylyl ether (Cetiol OE) and/or dicaprylyl carbonate, which may be obtained from Cognis for example under the trade name Cetiol CC, are advantageous.

It is also advantageous to select the oil component(s) from the group comprising isoeicosan, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethyl isosorbide. It is in particular advantageous if the oil phase of the formulations according to the invention has a C₁₂₋₁₅-alkyl benzoate content.

Advantageous oil components are also, e.g. butyloctyl salicylate (obtainable for example under the trade name Hallbrite BHB from CP Hall), tridecyl salicylate (which is obtainable under the trade name Cosmacol ESI from Sasol), C12-C15 alkyl salicylate (obtainable under the trade name Dermol NS from Alzo), hexadecyl benzoate and butyloctyl benzoate and mixtures thereof (Hallstar AB).

Any mixtures of such oil and wax components can also be used advantageously in the context of the present invention.

The oil phase can likewise also further contain advantageously non-polar oils, for example those which are selected from the group of branched and straight-chain hydrocarbons and waxes, especially mineral oil, vaseline (petrolatum), paraffin oil, squalene and squalene, polyolefins, hydrogenated polyisobutenes, C13-16 isoparaffin, C15-19 alkanes and isohexadecane. In terms of polyolefins, the preferred substances are polydecenes.

It is advantageous in accordance with the invention if the emulsion according to the invention contains ethanol and/or glycerol.

If the emulsion contains ethanol, a use concentration of 0.5 to 8% by weight, based on the total weight of the emulsion, is advantageous in accordance with the invention.

If the emulsion contains glycerol, a use concentration of 1 to 12% by weight, based on the total weight of the emulsion, is advantageous in accordance with the invention.

Advantageous embodiments of the present invention are also characterized in that the emulsion contains one or more active ingredients selected from the group UV filters, magnolia extract, glycyrrhetic acid, urea, arctiin, alpha-lipoic acid, folic acid, phytoene, D-biotin, coenzyme Q10, alpha-glucosylrutin, carnitine, carnosine, caffeine, natural and/or synthetic isoflavonoids, glycerylglucose, creatine, creatinine, taurine, tocopherol, tocopherol acetate, β-alanine and/or licochalcone A.

In accordance with the invention, advantageous UV filters may be selected, for example, from the group of the compounds 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene)camphor; 3-benzylidenecamphor; ethylhexyl salicylate; terephthalidenedicamphorsulfonic acid; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; homomenthyl salicylate; 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane-copolymer; 4-(tert-butyl)-4′-methoxydibenzoylmethane; hexyl hydoxybenzoyl)benzoate; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

It is advantageous according to the invention if the emulsion contains ethylhexylglycerin, propylene glycol, butylene glycol, 2-methylpropane-1,3-diol, 1,2-pentanediol, 1,2-hexanediol, 1,2-octanediol, piroctone olamine and/or 1,2-decanediol.

In accordance with the invention, advantageous embodiments of the present invention are characterized in that the emulsion contains phenoxyethanol and/or methylparaben. It is advantageous in accordance with the invention if the emulsion is free of propylparaben and butylparaben.

In accordance with the invention, advantageous embodiments are also characterized in that the emulsion contains at least 70% by weight water, based on the total weight of the emulsion.

### Comparative Test

With the following test, the effect according to the invention could be exemplified:

| | Sample A |
|---|---|
| Polyglyceryl-3 Methyglucose Distearate | 1.0 |
| Acrylates/C10-30 alkyl acrylate | 0.1 |
| crosspolymer | |
| Caprylic/Capric Triglyceride | 5 |
| Isopropyl Palmitate | 5 |
| Butyrospermum Parkii Butter | 1 |
| Dimethicone | 1 |
| Glycerol | 7 |
| 45% aqueous sodium hydroxide solution | 0.07 |
| Phenoxyethanol | 0.8 |
| Methylparaben | 0.3 |
| Aqua | 76.03 |
| Alcohol | 3 |

Spray Pattern Evaluation Sample A

9 bar positive pressure with nitrogen
valve DU 2527 from Aptar®
BOV—cup: Alu gold lacquered—inner gasket: Buna KA 6712—body valve: PP—spring: Inox 302—piston: POM-external gasket: butyl 1.2 mm foil: PET12/ALU8/OPA15/PP75.

| Spray distance | Spray pattern |
|---|---|
| 5 cm | uniformly round with ca. 5 cm diameter |
| 10 cm | uniformly round with ca. 6 cm diameter |
| 15 cm | uniformly round with ca. 10 cm diameter |

### EXAMPLES

The following examples are intended to clarify the present invention without limiting it. All quantitative data, fractions and percentages, unless otherwise stated, are specified based on the weight and the total amount or on the total weight of the preparations respectively.

| | B | C | D | E |
|---|---|---|---|---|
| | % | % | % | % |
| Polyglyceryl-3 Methylglucose | 1.0 | 1.5 | 0.7 | 0.6 |
| Distearate | | | | |
| Acrylates/C10-30 alkyl acrylate | 0.12 | 0.1 | 0.1 | 0.1 |
| crosspolymer | | | | |
| Xanthan Gum | | 0.05 | | 0.1 |
| Caprylic/Capric Triglyceride | 5 | 4 | 4 | 2 |
| Isopropyl Palmitate | 5 | | 6 | 3 |
| Butyrospermum Parkii Butter | 1 | | 0.5 | 1 |
| Dimethicone | 0.9 | | 1 | |
| C12-15 Alkyl benzoate | | | | 3 |
| Almond oil | 2 | | | |
| Dicaprylyl Ether | | 5 | | |
| Glycerol | 7 | 9 | 8 | 5 |
| Phenoxyethanol | 0.8 | 0.5 | 0.5 | 0.5 |
| Methylparaben | 0.3 | 0.2 | 0.3 | 0.3 |
| Cocoa butter | | 1 | | |
| Tocopherol acetate | | 0.5 | | |
| Alcohol | 3 | 1 | 2 | |
| Perfume | 0.3 | 0.35 | 0.3 | |
| Aqueous sodium | pH | pH | pH | pH |
| hydroxide solution | adjust- | adjust- | adjust- | adjust- |
| | ment | ment | ment | ment |
| Water | to 100 | to 100 | to 100 | to 100 |
| Initial pressure in bar | 8 | 9 | 7 | 8 |
| Pressurized gas | nitrogen | nitrogen | nitrogen | nitrogen |
| Spray valve | DU | DU | DU | DU |
| | 3520 | 3520 | 3527 | 3527 |

BOV applicator system, e.g. from Aptar®
Ex-EP BOV cup: Alu gold lacquered—inner gasket: Buna KA 6712—body valve: PP—spring: Inox 302—piston: POM-external gasket: butyl 1.2 mm foil: PET12/ALU8/OPA15/PP75

1.-15. (canceled)

16. A cosmetic spray, wherein the spray consists of
(a) an oil-in-water emulsion (O/W emulsion) comprising
(i) polyglyceryl-3 methylglucose distearate and
(ii) acrylate/C10-30 alkyl acrylate crosspolymer; and
(b) a spray applicator system.

17. The cosmetic spray of claim 16, wherein the spray applicator system is a bag-on-valve applicator system, in which a bag comprising the O/W emulsion is present in a pressurized gas container under positive pressure.

18. The cosmetic spray of claim 17, wherein the positive pressure in the pressurized gas container of the bag-on-valve applicator system is from 2 to 12 bar.

19. The cosmetic spray of claim 16, wherein the spray applicator features: 9 bar positive pressure with nitrogen valve DU 2527 or DU 3527 from Aptar®, BOV—cup: Alu gold lacquered—inner gasket: Buna KA 6712—body valve: PP—spring: Inox 302—piston: POM external gasket: butyl 1.2 mm foil: PET12/ALU8/OPA15/PP75.

20. The cosmetic spray of claim 16, wherein the bag containing the O/W emulsion is formed from a laminate of PE/adhesive/PA/adhesive/AL/adhesive/PET.

21. The cosmetic spray of claim 16, wherein the O/W emulsion comprises from 0.1% to 2% by weight of polyglyceryl-3 methylglucose distearate, based on a total weight of the emulsion.

22. The cosmetic spray of claim 16, wherein the emulsion comprises from 0.02% to 0.2% by weight of acrylate/C10-30 alkyl acrylate crosspolymer, based on a total weight of the emulsion.

23. The cosmetic spray of claim 16, wherein the emulsion further comprises caprylic/capric triglyceride.

24. The cosmetic spray of claim 16, wherein the emulsion further comprises isopropyl palmitate.

25. The cosmetic spray of claim 16, wherein the emulsion further comprises shea butter.

26. The cosmetic spray of claim 16, wherein the emulsion further comprises dimethicone and/or cyclomethicone.

27. The cosmetic spray of claim 16, wherein the emulsion further comprises ethanol.

28. The cosmetic spray of claim 16, wherein the emulsion further comprises glycerol.

29. The cosmetic spray of claim 16, wherein the emulsion further comprises one or more active ingredients selected from UV filters, magnolia extract, glycyrrhetic acid, urea, arctiin, alpha-lipoic acid, folic acid, phytoene, D-biotin, coenzyme Q10, alpha-glucosylrutin, carnitine, carnosine, caffeine, natural and/or synthetic isoflavonoids, glycerylglucose, creatine, creatinine, taurine, tocopherol, tocopherol acetate, β-alanine, licochalcone A.

30. The cosmetic spray of claim 16, wherein the emulsion further comprises one or more of propylene glycol, butylene glycol, 2-methylpropane-1,3-diol, 1,2-pentanediol, 1,2-hexanediol, 1,2-octanediol, 1,2-decanediol.

31. The cosmetic spray of claim 16, wherein the emulsion further comprises phenoxyethanol and/or methylparaben.

32. The cosmetic spray of claim 16, wherein the emulsion is free of propylparaben and ammonium acryloyldimethyl taurate/vinylpyrrolidone copolymer.

33. The cosmetic spray of claim 16, wherein the emulsion comprises at least 70% by weight water, based on a total weight of the emulsion.

34. A cosmetic spray, wherein the spray consists of
(a) an oil-in-water emulsion (O/W emulsion) comprising
(i) polyglyceryl-3 methylglucose distearate,
(ii) acrylate/C10-30 alkyl acrylate crosspolymer and
(iii) caprylic/capric triglyceride; and
(b) a spray applicator system.

35. The cosmetic spray of claim 34, wherein the O/W emulsion comprises from 0.1% to 2% by weight of polyglyceryl-3 methylglucose distearate, from 0.02% to 0.2% by weight of acrylate/C10-30 alkyl acrylate crosspolymer, and at least 70% by weight water, based on a total weight of the emulsion.
